(19) United States
(12) Patent Application Publication
(10) Pub. No.: US 2009/0248083 A1
(43) Pub. Date: Oct. 1, 2009

(54) ELONGATED CONNECTING ELEMENT WITH VARYING MODULUS OF ELASTICITY

(75) Inventors: Christopher M. Patterson, Olive Branch, MS (US); Eric C. Lange, Collierville, TN (US); Michael S. Veldman, Memphis, TN (US); Dimitri K. Protopsaltis, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/055,911

(22) Filed: Mar. 26, 2008

(51) Int. Cl. A61B 17/70

(52) U.S. Cl. 606/279; 606/246

(57) ABSTRACT

A spinal system comprising a spinal rod with an outer wall, a proximal end, a distal end, and a first axis extending centrally through the spinal rod between the proximal and the distal ends. The spinal rod comprises a first region having a first modulus of elasticity, a second region having a second modulus of elasticity different from the first modulus of elasticity, and a third region between the first and second region having a modulus gradation ranging from the first modulus of elasticity to the second modulus of elasticity.

### BACKGROUND

Elongated connecting elements such as rods, plates, tethers, wires, and cables are used to stabilize the spinal columns of patients with degenerative disc disease, vertebral fractures, scoliosis, and other degenerative or traumatic spine problems. In use, the elongated connecting elements may restrict or limit motion at a vertebral joint. Existing solutions have used a rigid or a flexible material to create elongated connecting elements with uniform properties throughout the length of the element. These systems may not provide sufficient ability to localize areas of rigidity and flexibility within a connecting element, and thus may not allow precise control of spinal motion.

### SUMMARY

In one embodiment, a spinal system comprises a spinal rod with an outer wall, a proximal end, a distal end, and a first axis extending centrally through the spinal rod between the proximal and the distal ends. The spinal rod comprises a first region having a first modulus of elasticity, a second region having a second modulus of elasticity different from the first modulus of elasticity, and a third region between the first and second region having a modulus gradation ranging from the first modulus of elasticity to the second modulus of elasticity.

In another embodiment, a spinal rod comprises a first region with a first modulus of elasticity and a second region with a second modulus of elasticity. The rod further includes a transition region between the first region and the second region, the transition region having variations in moduli of elasticity.

In another embodiment, a method of using a spinal rod comprises connecting a spinal rod with a first connector to a first vertebral member and with a second connector to a second vertebral member. The spinal rod includes first and second rigid regions, a central region between the first and second regions, and transition regions between the central region and each of the first and second regions. The central region is more flexible than the first and second regions. The method further includes positioning the first region of the spinal rod at the first connector and positioning the second region of the spinal rod at the second connector.

Additional and alternative features, advantages, uses and embodiments are set forth in or will be apparent from the following description, drawings, and claims.

### BRIEF DESCRIPTION OF THE DRAWINGS

FIG. 1 is a perspective view of a vertebral joint with a vertebral stabilization system according to one embodiment.

FIGS. 2a, 2b, 3a, and 3b are perspective views of elongated connecting elements according to embodiments of this disclosure.

FIGS. 4a, 4b, 5a, and 5b are cross-sectional views of elongated connecting elements according to embodiments of this disclosure.

FIG. 6a is a perspective view of an elongated connecting element with a reinforcement member.

FIG. 6b is a cross-sectional view of the elongated connecting element of FIG. 6a.

FIGS. 7-8 are perspective views of elongated connecting elements with reinforcement members according to other embodiments of this disclosure.

FIGS. 9a and 9b are sectional views of the reinforcement members of FIG. 8 in unloaded and loaded states.

FIG. 10 is a sectional view of a reinforcement member according to an embodiment of the disclosure.

### DESCRIPTION

The present disclosure relates generally to systems and methods for spinal surgery and, more particularly in some embodiments, to spinal connection elements which may have localized differences in stiffness. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

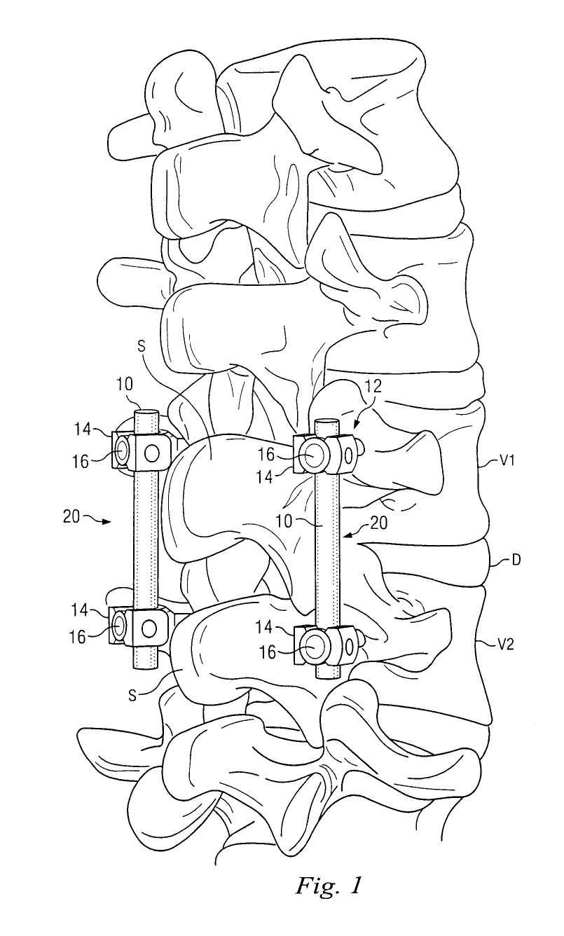

Referring first to FIG. 1, one type of elongated connecting element system, a spinal rod system, is indicated generally by the numeral 20. Various specific embodiments of the spinal rod system will be described in detail below. FIG. 1 shows a perspective view of first and second spinal rod systems 20 in which spinal rods 10 are attached to vertebral members V1 and V2. A vertebral disc D extends between vertebral members V1, V2 and together these structures define a vertebral joint. The system 20 may also be used if all or a portion of disc D has been removed and replaced with a fusion or motion preserving implant. In the example systems 20 shown, the rods 10 are positioned at a posterior side of the spine, on opposite sides of the spinous processes S. In alternative embodiments, spinal rods 10 may be attached to a spine at other locations, including lateral and anterior locations. Spinal rods 10 may also be attached at various sections of the spine, including the base of the skull and to vertebrae in the cervical, thoracic, lumbar, and sacral regions. Thus, the illustration in FIG. 1 is provided merely as a representative example of one application of a spinal rod 10.

In the exemplary system 20, the spinal rods 10 are secured to vertebral members V1, V2 by connector assemblies 12 comprising a pedicle screw 14 and a retaining cap 16. The outer surface of spinal rod 10 is grasped, clamped, or otherwise secured between the pedicle screw 14 and retaining cap 16. Other mechanisms for securing spinal rods 10 to vertebral members V1, V2 include hooks, cables, and other such devices. Further, examples of other types of retaining hardware include threaded caps, screws, and pins. Spinal rods 10 are also attached to plates in other configurations. Thus, the exemplary assemblies 20 shown in FIG. 1 are merely representative of one type of attachment mechanism.

For the present discussion, an exemplary elongated connecting element is described as a rod, but other elements and structures may be used, such as a plate, hollow cylinder, blocks, discs, etc., without departing from the spirit and scope of the invention. The invention is not limited to a rod and is limited only by the claims appended hereto. Moreover, if a rod is used, it is not limited to a circular cross section, but may have an oval, rectangular, hexagonal, or any other regular or irregular cross section shape without departing from the spirit and scope of the invention. The rods may have substantially uniform circular cross-sectional areas along the longitudinal axis, but in alternative embodiments, the size and/or shape of the cross sectional area may vary along the length of the longitudinal axis. The rod may be curved, non-curved, or capable of being curved, depending on the circumstances of each application.

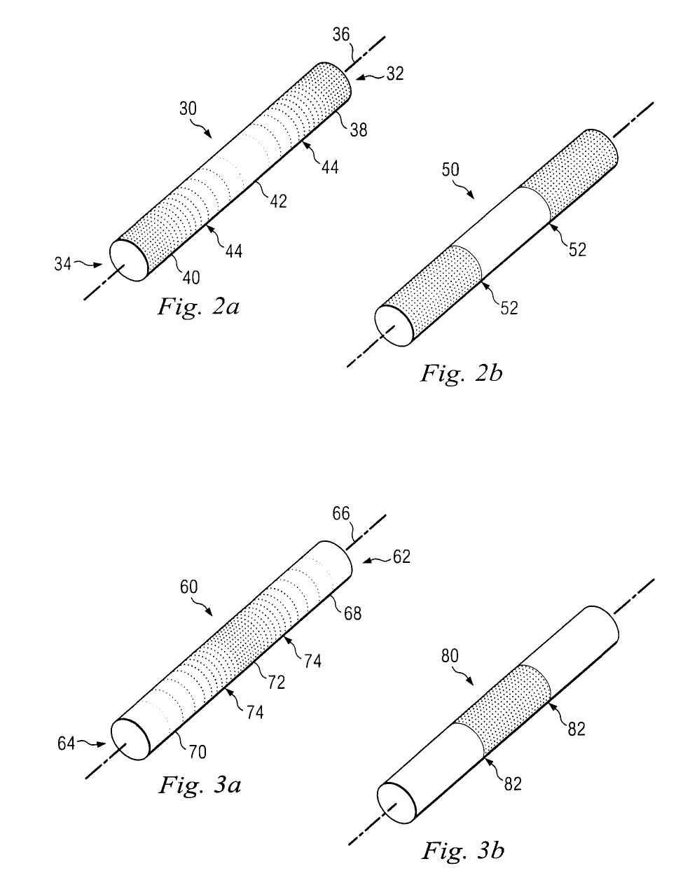

Referring now to FIG. 2a, in one embodiment, a spinal rod 30 may be used as the rod of the spinal system 20. The spinal rod 30 includes a proximal end 32, a distal end 34, and a longitudinal axis 36 extending centrally through the rod between the proximal and distal ends. The rod 30 has regions of differing moduli of elasticity. Throughout this disclosure, areas with higher moduli of elasticity will be indicated with shading darker than areas of low elastic modulus. Such shading is representative only, and it is understood that an actual rod may not have any visually perceptible indications of flexibility or rigidity. All shading or stippling is merely representative of degree of modulus of elasticity and is not intended to necessarily indicate concentration of particulate matter. In FIG. 2a, the rod 30 includes a region 38 located at the proximal end 32 and a region 40 located at the distal end 34 which have a higher modulus of elasticity, and thus are more rigid, than a central region 42. Greater rigidity at the end regions 38, 40 may allow a more secure connection between the rod 30 and the connector assemblies 12. As installed, the lower modulus central region 42 may be located proximate to the area of disc D to allow more stretching and compression of the rod 30 when the vertebral joint is in motion. In this embodiment, the rod 30 also includes transition regions 44 having a modulus gradation, and thus a gradual transition, between the higher moduli of the regions 38, 40 and the lower modulus of the central region 42.

Referring now to FIG. 2b, in this embodiment, a spinal rod 50 may be used as the rod of the spinal system 20. The rod 50 may be substantially similar to rod 30 but includes the following difference. The spinal rod 50 includes transition regions 52 in which an abrupt or discrete change occurs between the more rigid end regions and the more flexible central region.

Referring now to FIG. 3a, in another embodiment, a spinal rod 60 may be used as the rod of the spinal system 20. The spinal rod 60 includes a proximal end 62, a distal end 64, and a longitudinal axis 66 extending centrally through the rod between the proximal and distal ends. The rod 60 also has regions of differing moduli of elasticity. For example, the rod 60 includes a region 68 located at the proximal end 62 and a region 70 located at the distal end 64 which have a lower modulus of elasticity than a central region 72 which is more rigid. Greater rigidity along the central region 72 may allow the rod 60 to be more resilient to outside forces that might otherwise be damaging to the spinal system or the vertebral joint. As installed, the higher modulus central region 72 may be located proximate to the area of disc D to provide more resistance to vertebral joint motion. In this embodiment, the rod 60 also includes transition regions 74 having a modulus gradation, and thus a gradual transition, between the higher moduli of the central region 72 and the lower moduli of the end regions 68, 70.

Referring now to FIG. 3b, in this embodiment, a spinal rod 80 may be used as the rod of the spinal system 20. The rod 80 may be substantially similar to rod 60 but includes the following difference. The spinal rod 80 includes transition regions 82 in which an abrupt or discrete change occurs between the more rigid central region and the more flexible end regions.

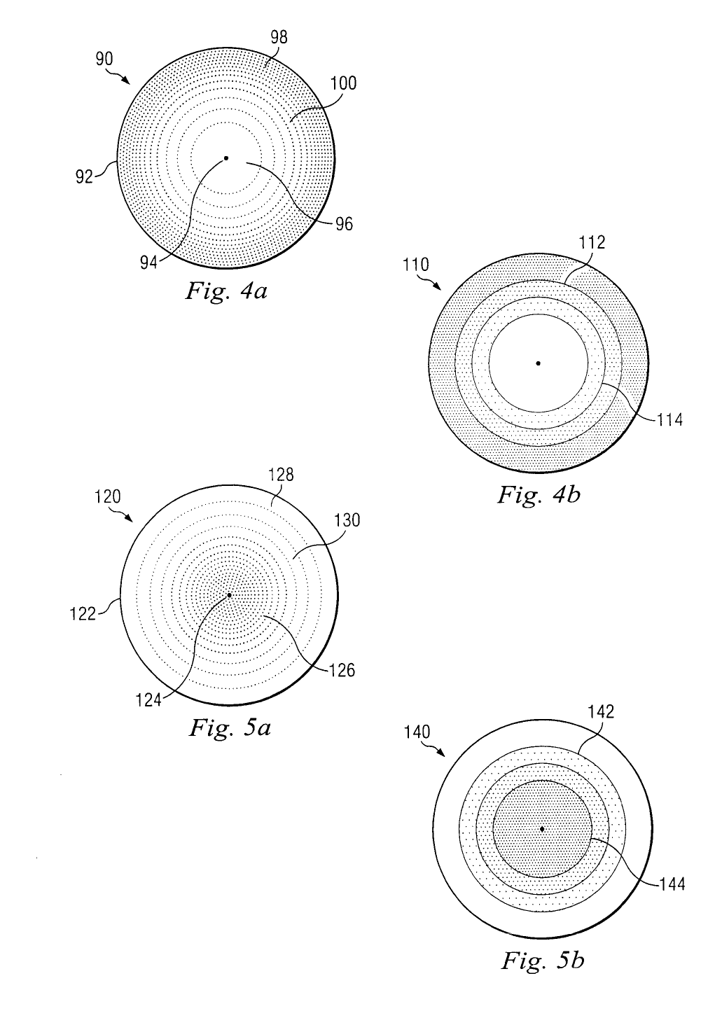

Referring now to FIG. 4a, in this embodiment, a spinal rod 90 may be used as the rod of the spinal system 20. The rod 90 has an outer wall 92 and a shape substantially similar to the elongated shape of rod 30. Like the axis 36 of rod 30, rod 90 has a longitudinal axis 94 extending through the rod between proximal and distal ends. A center region 96 extends along the longitudinal axis 94. An outer region 98 extends along the outer wall 92. In this embodiment, the outer region 98 has a higher modulus of elasticity than the center region 96, and thus the outer region of the rod is more rigid than the center region along the longitudinal axis. A transition region 100 extends between the outer region and the center region. The transition region 100 has a modulus gradation, and thus a gradual transition, between the higher moduli of the region 92 and the lower modulus of the region 96.

Referring now to FIG. 4b, in this embodiment, a spinal rod 110 may be used as the rod of the spinal system 20. The rod 110 may be substantially similar to rod 90 but includes the following difference. The spinal rod 110 includes transition regions 112, 114 which provide abrupt or discrete change in modulus of elasticity between the more rigid outer region and the more flexible center region. These transition regions create discrete tubular, band-like rings about the longitudinal axis of the rod 110.

Referring now to FIG. 5a, in this embodiment, a spinal rod 120 may be used as the rod of the spinal system 20. The rod 120 has an outer wall 122 and a shape substantially similar to the elongated shape of rod 30. Like the axis 36 of rod 30, rod 120 has a longitudinal axis 124 extending through the rod between proximal and distal ends. A center region 126 extends along the longitudinal axis 124. An outer region 128 extends along the outer wall 122. In this embodiment, the outer region 128 has a lower modulus of elasticity than the center region 126, and thus the center along the longitudinal axis is more rigid. A transition region 130 extends between the outer region and the center region. The transition region 130 has a modulus gradation, and thus a gradual transition, between the lower moduli of the region 122 and the higher modulus of the region 126.

Referring now to FIG. 5b, in this embodiment, a spinal rod 140 may be used as the rod of the spinal system 20. The rod 140 may be substantially similar to rod 120 but includes the following difference. The spinal rod 140 includes transition regions 142, 144 which provide abrupt or discrete change in modulus of elasticity between the more flexible outer region and the more rigid center region. These transition regions create discrete tubular, band-like rings about the longitudinal axis of the rod 140.

In alternative embodiments, a spinal rod may combine the properties of any of the rods 30, 50, 60, 80 with the rods 90, 110, 120, 140. That is, the modulus of elasticity may vary both along the longitudinal axis and from the longitudinal axis to the outer wall of the rod. For example, a spinal rod may have a rigid core and softer regions at the ends and near the outer surface area of the rod. Alternatively, a spinal rod may have a softer interior, near the midpoint of the length of the rod, and may have more rigid ends and outer surface area. In still further alternative embodiments, a rod may have a series of rigid, transition, and flexible regions along the length of the rod which may be particularly suitable if a rod spans multiple vertebral joints.

Each of the above described spinal rods may be formed of a common base material throughout all of the regions. Suitable base materials may include polymers, ceramics, or metals. The selected material may allow the rod to stretch, compress, and laterally bend. Example materials may include shape memory alloys or shape memory polymers. Suitable elastomeric materials may include polyurethane, silicone, silicone polyurethane copolymers, polyolefins, such as polyisobutylene rubber and polyisoprene rubber, neoprene rubber, nitrile rubber, vulcanized rubber and combinations thereof. Other polymers such as polyethylene, polyester, and polyetheretherketone (PEEK), polyaryletherketone (PAEK), or polyetherketone (PEK) may also be suitable.

Both the modulus gradation described for rods 30, 60, 90, and 120 and the abrupt modulus transition described for rods 50, 80, 110, and 140 may be achieved through molding methods. For example, multishot molding would allow each of the regions to be formed in progressive stages. Because a common base material may be used, adhesion problems between the molded layers may be minimized. The common base material may be chemically treated, altered by physical forces such as pressure or temperature, or supplemented with additional material to create the regions of differing modulus. The modulus transition, particularly the more gradual modulus transition of the rods 30, 60, 90, and 120 may be created by varying the amount and type of chemical crosslinking. Alternatively, the modulus transition may be created by a chemical reaction such as the injection of a catalyst to change the material properties of the injected location. For example, the injection of isocyanate into a region in a base material of polyurethane can alter the stiffness of the injected region. Gradient changes may also result from combining or dispersing additional materials in varying amounts throughout the otherwise homogeneous base material to achieve a desired combined or blended modulus.

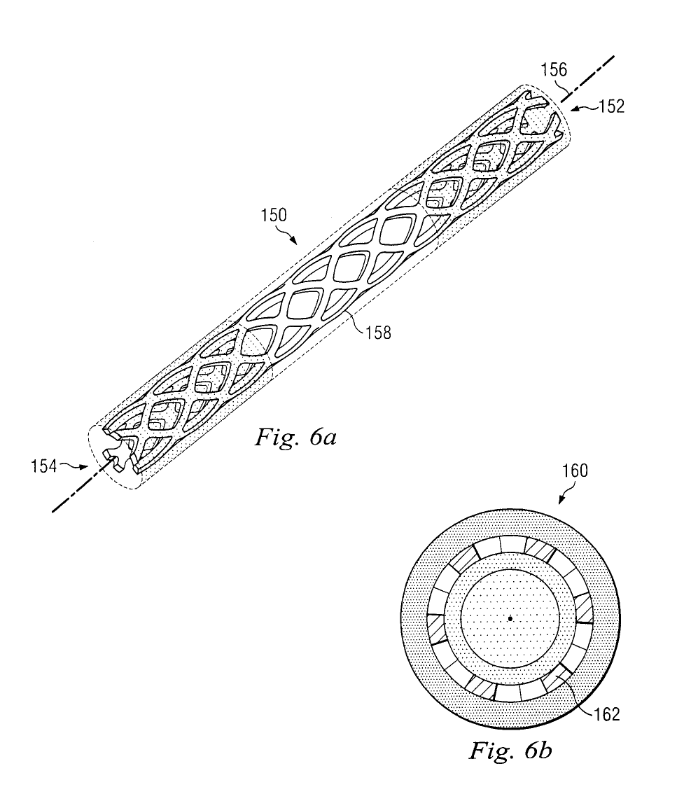

Referring now to FIG. 6a, in this embodiment, a spinal rod 150 may be used as the rod of the spinal system 20. The rod 150 may be substantially similar to rod 30 including a rigid proximal end 152, a rigid distal end 154, and a longitudinal axis 156 extending between the ends. The rod 150 further includes a reinforcement member 158. In this embodiment, the reinforcement member 158 may be a textile or fabric formed of braided or woven fibers and configured as a tubular sleeve extending about the axis 156 from the proximal end 152 to the distal end 154. The reinforcement member may limit the amount the rod 150 may both stretch and compress. Further, the reinforcement member 158 may increase the resistance of the rod 150 to tensile and shear forces. The reinforcement member 158 may be integrally molded or inserted into the body of the rod. In alternative embodiments a reinforcement member may be used only in selected regions of the rod.

Referring now to FIG. 6b, in this embodiment, a spinal rod 160 may be used as the rod of the spinal system 20. The rod 160 may have a series of discrete layered regions having a common base material, similar to the rod 110. The rod 160 may include a reinforcement member 162 substantially similar to the reinforcement member 158 extending between outer and center regions of the rod. The rod 160 may be formed by extending the tubular reinforcement member 160 around an initially molded center region. The outer region may then be molded or extruded over the reinforcement member.

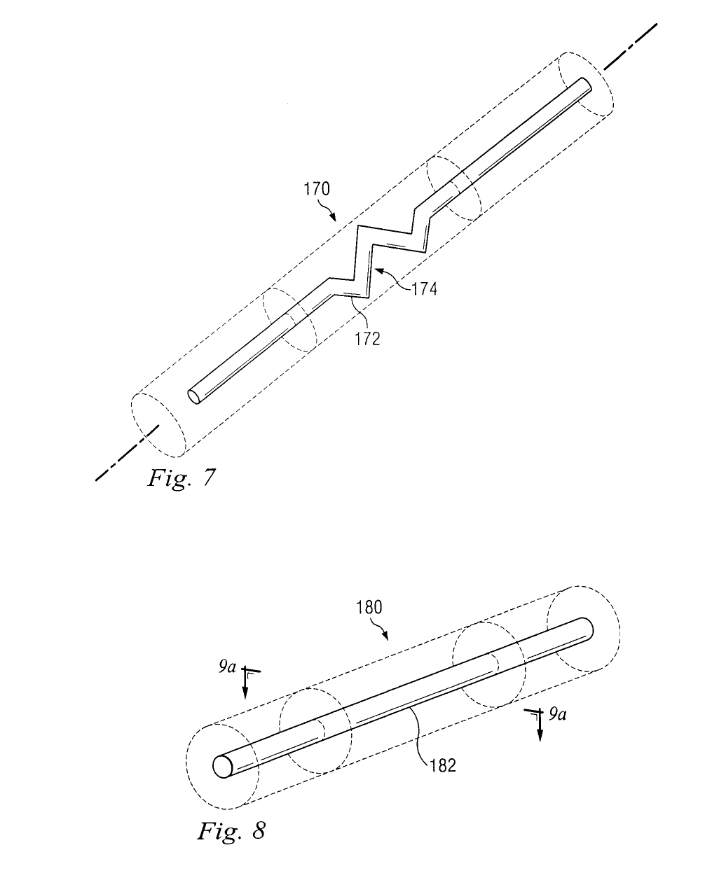

Referring now to FIG. 7, in this embodiment, a spinal rod 170 may be used as the rod of the spinal system 20. The rod 170 may be similar to rod 150 but including a reinforcement member 172 extending between proximal and distal ends. In this embodiment the reinforcement member 172 may be a tether integrated into the rod 170 to resist tensile forces and prevent overstretching. The reinforcement member 172 may be formed from a plurality of fibers or may be a unitary structure. As shown, the reinforcement member 172 may have a bent or corrugated region 174 that may allow the rod to stretch as the bent region becomes straightened under a tensile or lateral bending load. As the reinforcement member becomes straightened and reaches its elastic limit, the reinforcement member may limit further stretching or bending of the rod 170. The reinforcement member 172 with the bent region 174 may also provide compression resistance.

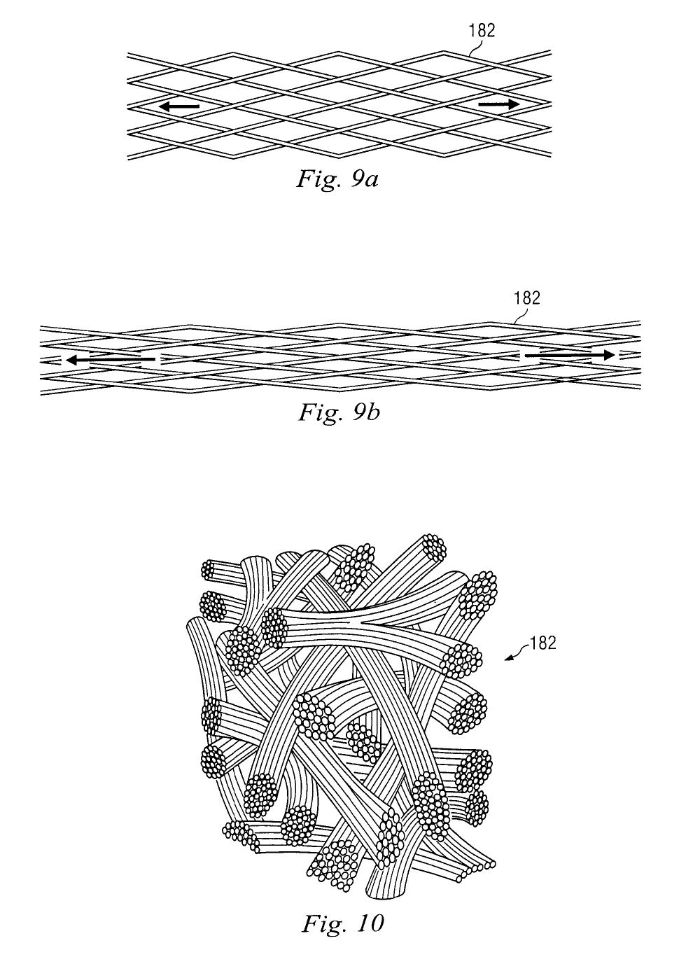

Referring now to FIGS. 8-10, in this embodiment, a spinal rod 180 may be used as the rod of the spinal system 20. The rod 180 includes a reinforcement member 182 extending between proximal and distal ends of the rod. In this embodiment the reinforcement member 182 may be a tether formed of folded, crimped, or wave-like fibers, similar to collagen. The fibers may be intertwined as shown in FIG. 10. As shown in simplified FIGS. 9a-9b, when the reinforcement member 182 is subjected to a tensile load, the fibers are unfolded and the tether elongates to the limit permitted by the fibers. The reinforcement member 182 thus allows the rod 180 to resist excessive tensile forces and strengthens the rod against shear forces.

The reinforcement members of FIGS. 6a-10 may be formed of any suitable natural or synthetic fibers or solids including ultra high molecular weight polyethylene (UHMWPE) fibers, polyethylene terephthalate (PET) fibers, polyester fibers, or metallic fibers.

The non-elastic polymers may be incorporated in the form of fibers, non-woven mesh, woven fabric, or a braided structure.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A spinal system comprising:
a spinal rod with an outer wall, a proximal end, a distal end, and a first axis extending centrally through the spinal rod between the proximal and the distal ends, the spinal rod comprising a first region having a first modulus of elasticity, a second region having a second modulus of elasticity different from the first modulus of elasticity, and a third region between the first and second region having a modulus gradation ranging from the first modulus of elasticity to the second modulus of elasticity.

2. The spinal system of claim 1 wherein the first region is located at the proximal end and the second region is located between the proximal end and the distal end.

3. The spinal system of claim 2 wherein the first modulus is greater than the second modulus.

4. The spinal system of claim 2 wherein the second modulus is greater than the first modulus.

5. The spinal system of claim 1 wherein the first region is along the first axis and the second region is along the outer wall.

6. The spinal system of claim 5 wherein the first modulus is greater than the second modulus.

7. The spinal system of claim 5 wherein the second modulus is greater than the first modulus.

8. The spinal system of claim 2 further comprising a fourth region having a modulus approximately the same as the first modulus, the fourth region located at the distal end.

9. The spinal system of claim 1 wherein the spinal rod comprises a common base material extending from the proximal end to the distal end.

10. The spinal system of claim 9 wherein the third region includes a plurality of layers, each of the plurality of layers comprising the base material and having a different modulus of elasticity than the other of the plurality of layers.

11. The spinal system of claim 1 further comprising a fibrous reinforcement material between the first region and the second region.

12. The spinal system of claim 1 further comprising a fibrous reinforcement material extending from the proximal end to the distal end.

13. The spinal system of claim 12 wherein the fibrous reinforcement material extends around the first axis.

14. The spinal system of claim 1 further comprising a non-fibrous reinforcement material extending from the proximal end to the distal end.

15. The spinal system of claim 1 wherein the spinal rod has a uniform cross-sectional area between and including the proximal end and the distal end.

16. The spinal system of claim 1 wherein the spinal rod has a circular cross section.

17. The spinal system of claim 1 further comprising a connector for attaching the spinal rod to a vertebra.

18. A spinal rod comprising:
a first region with a first modulus of elasticity;
a second region with a second modulus of elasticity;
a transition region between the first region and the second region, the transition region having variations in moduli of elasticity.

19. The spinal rod of claim 18 wherein the first and second regions are more rigid than the transition region.

20. The spinal rod of claim 18 wherein the transition region includes an abrupt variation in moduli.

21. The spinal rod of claim 18 wherein the transition region includes a gradual variation in moduli.

22. A method of using a spinal rod, the method comprising:
connecting a spinal rod with a first connector to a first vertebral member and with a second connector to a second vertebral member, the spinal rod including first and second rigid regions, a central region between and more flexible than the first and second regions, and transition regions between the central region and each of the first and second regions;
positioning the first region of the spinal rod at the first connector; and
positioning the second region of the spinal rod at the second connector.

23. The method of claim 22 wherein the transition regions includes a gradual change in modulus of elasticity.

24. The method of claim 22 wherein the transition regions includes an abrupt change in the modulus of elasticity.

25. The method of claim 22 wherein the spinal rod includes a common base material in the first, second, central, and transition regions.
